(19) United States
(12) Patent Application Publication
(10) Pub. No.: US 2004/0096450 A1
(43) Pub. Date: May 20, 2004

(54) Methods and compositions for treating diseases associated with increased Fas-ligand titers

(76) Inventors: Lars E. French, Genf (CH); Isabelle Viard, Vetraz-Monthoux (FR); Jurg Tschopp, Epalinges (CH)

Correspondence Address: FISH & RICHARDSON PC, 225 FRANKLIN ST, BOSTON, MA, 02110, US

(21) Appl. No.: 10/706,400

(22) Filed: Nov. 11, 2003

Related U.S. Application Data
(63) Continuation of application No. 09/419,262, filed on Oct. 12, 1999.

(30) Foreign Application Priority Data
Jul. 5, 1999 (WO) PCT/EP99/04655
Jan. 8, 1999 (DE) 19900503.6

(51) Int. Cl. G01N 33/53; A61K 39/395

(52) U.S. Cl. 424/146.1; 435/7.1

(57) ABSTRACT

The invention relates to methods and compositions for the treatment of human or animal disorders associated with pathophysiologically based extracellular Fas ligand titers, as well as methods for determining the prophylactic suitability and quality control of compositions, such as intravenous immunoglobulin mixtures, for use in such methods, and methods of preparing compositions for treating such disorders.

### CROSS REFERENCE TO RELATED APPLICATIONS

This application claims benefit of priority from PCT/EP99/04655, filed on Jul. 5, 1999, and German Patent Application No. 19900503.6, filed on Jan. 8, 1999, both of which are incorporated herein by reference in their entirety.

### FIELD OF THE INVENTION

The invention relates to methods and compositions for the treatment of illnesses associated with increased extracellular Fas-ligand titers.

### BACKGROUND OF THE INVENTION

To maintain physiological homeostasis, a control of cell proliferation is required that is differentiated precisely and in detail. The control of cell proliferation in living organisms is possible through the mechanism of programmed cell destruction (apoptosis). Numerous signal transduction channels are well-known from the existing literature; these lead in the end to programmed cell-destruction. Here the interaction of extracellular ligands, e.g., the Fas-ligand (FasL), with diverse cell surface receptors, e.g., the Fas receptor (Fas), or the TRAIL receptor, plays a central role in causing this apoptosis. In diverse scientific works, a connection has been established between the appearance of different illnesses and a causative, pathological failure to regulate apoptosis (see, e.g., Steller, Science, 267, 1445-1449, 1995; Thompson, Science, 267, 1456-1462, 1995; Nagata, Cell, 88, 355-365, 1997; Giordano et al., Science, 275, 960-963, 1997; and Chervonsky et al., Cell, 89, 17-24, 1997). Both the excessive apoptotic reaction and the loss of extracellular apoptotic signals lead to pathophysiological conditions, illnesses, or health disturbances in living organisms. In many cases the exact etiology of the apoptotic failed function is not clear.

Increased apoptosis of target tissues has been implicated in various disorders, including graft-versus-host-disease (GVHD), which is a serious disease that results from the attack by transplanted donor lymphocytes against the host's skin, liver, and gut. Although in its aggressive form, GVHD can be a serous life-threatening disease, in its mild form, the disease can be useful due to its anti tumor effect.

GVHD is caused by the reaction of donor T-cells against host histocompatibility antigens, and is the major complication resulting from bone marrow transplantations. Clinical GVHD of varying severity affects 30-50% of marrow allograft recipients, although its incidence and severity can be somewhat limited by careful donor matching, T-cell depletion of bone marrow before transplantation, prophylaxis by immunosuppressive agents (cyclosporine among others), and treatment of established GVHD with steroids and other immunosuppressors. Donor T-cells are thought to initiate the disease, and increased apoptosis of target tissues has been clearly demonstrated, but the pathogenesis of immune-mediated skin, gut and liver damage is still incompletely understood. Indeed, the relative importance of cytokines compared to direct killing by cells, including cytolytic T-lymphocytes (CTL), macrophages or NK cells remains controversial.

### SUMMARY OF THE INVENTION

The invention concerns the use of compositions for the manufacture of drugs to treat human or animal conditions, illnesses, or health disturbances associated with pathophysiologically increased extracellular FasL titers (soluble FasL and/or membrane-bound FasL); processes for determining the prophylactic suitability and quality control in vitro of a composition for its later use in the manufacture of a drug for the treatment of the above-mentioned disorders; and processes for the manufacture of a drug with increased pharmaceutical effectiveness for the treatment of the above-named disorders.

It is the intention of the present invention to identify pathophysiological conditions, illnesses, and health disturbances that are based on an increased apoptotic activity, and to develop a suitable, goal-oriented pharmaceutical treatment to deal with this kind of impaired regulation that takes causation into account and that has an inhibiting effect on the excessive apoptotic reaction. It is also the intention of the present invention to identify those parts of substance mixtures, used so far for unspecific and partially unsuccessful treatment, that cause therapeutic success.

The invention furthermore aims at the development of a process for suitability and quality control of such unspecific substance mixtures that allow an unlimited successful medicinal application. It is also a goal for the invention to improve such substance mixtures in their pharmaceutical effectiveness so that they may show an increased pharmaceutical effectiveness for the treatment of the above-named illnesses, health disturbances, or pathophysiological conditions.

In general, the invention features a method of treating a subject having a disorder associated with increased extracellular Fas ligand titers by administering to the subject a composition including anti-Fas antibodies, e.g., an intravenous immunoglobulin (IVIG) mixture, in an amount effective to inhibit binding of Fas ligands to Fas receptors in the subject. The method can be used to treat, e.g., toxic epidermal necrolysis (Lyell's Syndrome), graft-versus-host disease (GVHD), hepatitis, fulminant hepatitis, autoimmune thyroiditis (Hashimoto's thyroiditis), malignant tumor illnesses (e.g., melanoma), or HIV. The composition can contain a level of anti-Fas antibodies sufficient to inhibit at least 40, 50, 60 percent, or more, of FasL binding to Fas receptors. The composition can be administered at a dosage of at least 0.1 to 0.75 g/kg/day or more, e.g., by infusion or repeated bolus injections.

In another aspect, the invention features a method of treating a subject having graft-versus-host-disease (GVHD) by administering to the subject a composition (e.g., a human IVIG mixture) including anti-Fas antibodies in an amount effective to inhibit binding of Fas ligands to Fas receptors in the subject. The IVIG can contain an anti-Fas antibody at a concentration of at least 0.1 to 8.0 mg/ml, or more. The composition can include an anti-Fas antibody administered at a dosage of at least 0.1 to 0.5 mg/kg/day, or more, for at least two, three, or four days, or more. An IVIG mixture can be administered, e.g., by infusion, at a dosage of 0.75 g/kg/day, or more, for one, two, three, four, or more consecutive days.

The invention further includes a method for determining the prophylactic suitability and quality control of a composition (e.g., an IVIG mixture) for use in treating a disorder associated with increased extracellular Fas ligand titers by (a) incubating the composition with a Fas-Fc fusion protein in a solution; (b) adding to the solution a marked Fas ligand; and (c) detecting the amount of Fas ligand bound to the Fas-Fc fusion protein as an indication of the presence of anti-Fas antibodies in the composition, wherein an amount of anti-Fas antibodies in the composition sufficient to inhibit binding of Fas ligand to Fas receptor indicates that the composition is suitable for use in treating a disorder associated with increased extracellular Fas ligand titers. The percentage of binding inhibition can be at least 40, 50, 60 percent or more.

In another aspect, the invention features a method for determining the prophylactic suitability and quality control of a composition for use in treating a disorder associated with increased extracellular Fas ligand titers by (a) incubating Fas sensitive cells with the composition in a solution; (b) adding soluble Fas ligand to the solution; and (c) determining the percentage of Fas sensitive cells in which apoptosis is inhibited compared to cells not incubated with the composition, wherein a composition that inhibits apoptosis of Fas sensitive cells is suitable for use in treating a disorder associated with increased extracellular Fas ligand titers.

The invention also features a method for determining the prophylactic suitability and quality control of a composition for use in treating a disorder associated with increased extracellular Fas ligand titers, by (a) combining Fas receptors with the composition; (b) adding labelled secondary antibodies that bind specifically to anti-Fas antibodies; and (c) detecting the labelled secondary antibodies as an indication of the presence of anti-Fas antibodies bound to the Fas receptors, wherein the presence of anti-Fas antibodies in the composition indicates that the composition is suitable for use in treating a disorder associated with increased extracellular Fas ligand titers. The Fas receptors and the composition can be combined in a Western blot technique.

In another embodiment, the invention features a method of preparing a drug to treat disorders associated with increased extracellular Fas ligand titers by (a) fractionating a composition; (b) examining each fraction to determine the presence of anti-Fas antibodies; (c) isolating each fraction that contains anti-Fas antibodies; and (d) concentrating the isolated fractions for use as the drug. The method can further include (e) purifying and isolating the anti-Fas antibodies in the isolated fractions by affinity chromatography. The affinity chromatography can include the use of column chromatography using Fas fusion proteins bound to the column. In addition, the affinity chromatography can include the use of one or more chromatographic columns, each column having linked thereto a specific amino acid sequence of the Fas fusion protein that corresponds to a specific Fas antibody epitope, wherein all Fas antibody epitopes are bound to the one or more columns and are then eluted.

In yet another embodiment, the invention includes a composition for the treatment of disorders associated with increased extracellular Fas ligand titers, the composition including anti-Fas antibodies that inhibit binding of Fas ligand to the Fas receptor. The anti-Fas antibodies can be of human or non-human origin and be humanized. The composition can be an IVIG mixture from pooled human blood serum.

An effective amount of a composition of the invention is an amount of a Fas-antibody containing composition, e.g., an immunoglobulin (Ig) mixture, that is sufficient to inhibit the binding of Fas ligands to Fas receptors in a subject by a measurable amount. The effective amount of a composition of the present invention will vary with the particular disorder (e.g., GVHD) being treated, the age and physical condition of the subject being treated, the severity of the disorder, the duration of treatment, the nature of concurrent therapy, the specific form of the antibody composition employed, the particular vehicle or carrier, and like factors within the knowledge and expertise of the attending physician.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINQS

The drawings are first briefly described.

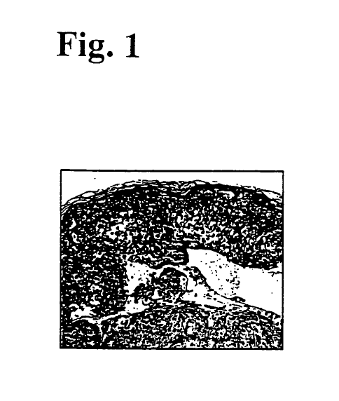

FIG. 1 is a photomicrograph of a skin tissue sample from a toxic epidermal necrolysis (TEN) patient. The epidermis has been displaced from the epidermis underneath it, a characteristic pathological phenomenon in TEN patients.

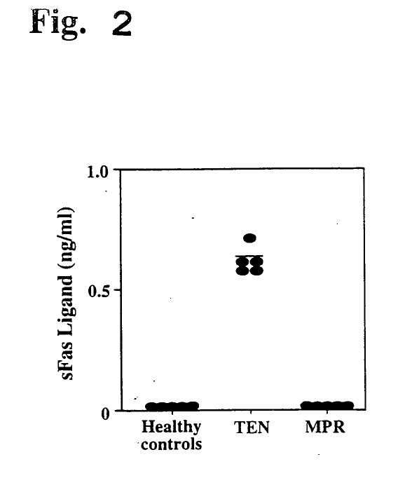

FIG. 2 is a graph documenting the characteristically increased serum titers of soluble FasL (sFasL) in TEN patients. The sFasL concentrations are above 0.5 ng/ml in the serum samples. On the other hand, the serum concentrations of healthy control patients and patients with another skin condition, macular-popular skin rash (“MPR”), negative control, have insignificant sFasL titers.

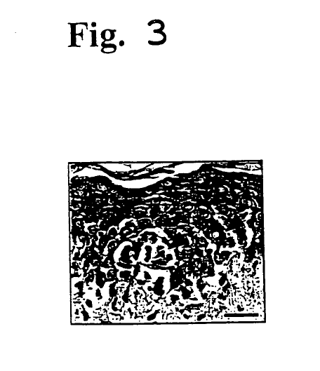

FIG. 3 is a photomicrograph of a tissue sample of the skin of TEN patients according to histological evaluation. The arrows indicate apoptotic keratinocytes.

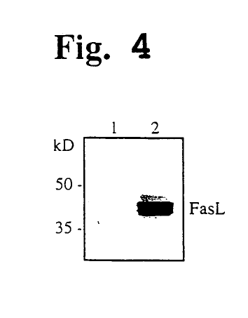

FIG. 4 is a representation of an immunoblot of monoclonal antibodies directed against FasL. Lane 1 contains a 293 pseudo-transferred cell lysate, Lane 2 contains a 293 transferred cell lysate. The antibody reaction is specifically directed against the correspondingly transferred cells.

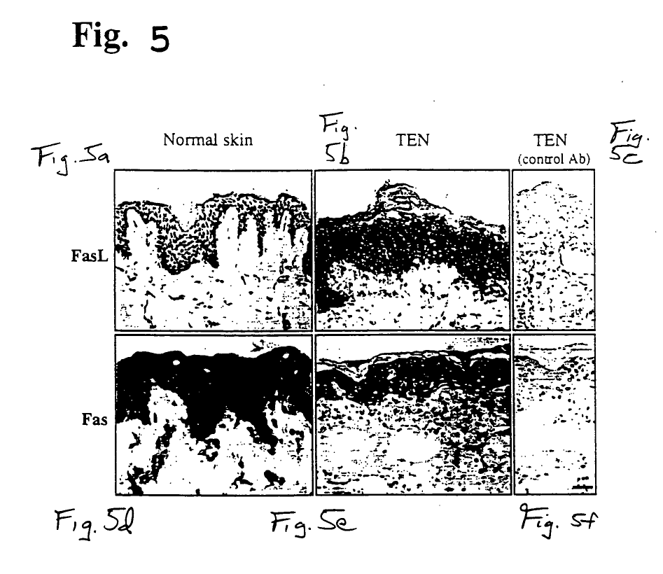

FIGS. 5a-5f are photomicrographs of skin tissue samples that have been immunohistochemically examined. The samples of skin tissues of healthy persons (FIGS. 5a and d) and from TEN patients (FIGS. 5b, c, e, and f) have been treated with anti-Fas antibodies (FIGS. 5a and b, Anti-FasL; FIGS. 5d and e, Anti-Fas). As a control, control antibodies have been used that exhibit an unspecific antibody reaction (FIGS. 5c and f). FIG. 5b shows a strong antibody reaction against FasL in the epidermis of TEN patients.

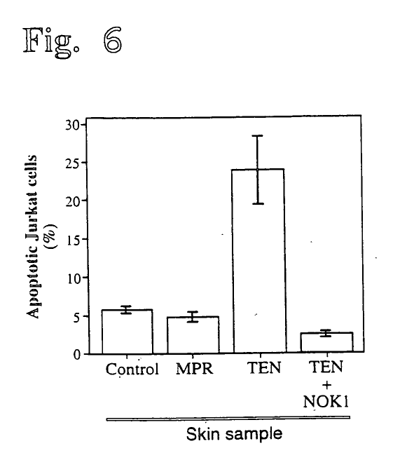

FIG. 6 is a bar graph that reflects the result (with statistical error bars) of experiments with Fas sensitive Jurkat cells that have been stratified on cryogenic skin segments from healthy, TEN, and MPR patients. The bars in the graph show the percentage of apoptotic Jurkat cells in each case after a 6 hour incubation, as determined by fluid cytometry. The tissues of TEN patients incite a significantly increased apoptotic reaction. These are strongly diminished by FasL blocking antibodies (NOK1) (4th bar in graph) (control).

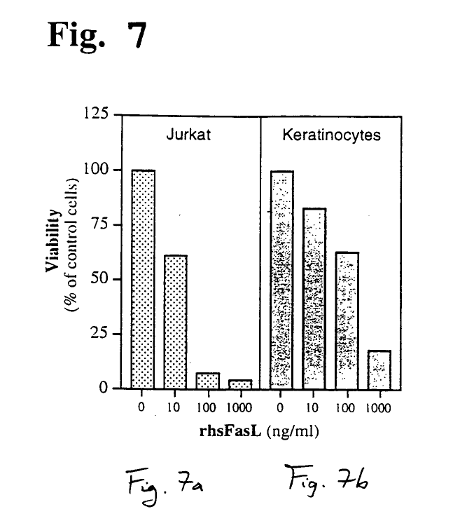

FIGS. 7a and 7b are bar graphs showing the viability of Jurkat cells (7a) and primary human keratinocytes (7b) in reference to rhsFasL (Alexis Corporation) after 6 and 16 hour incubation. Here, keratinocytes are shown to be FasL sensitive.

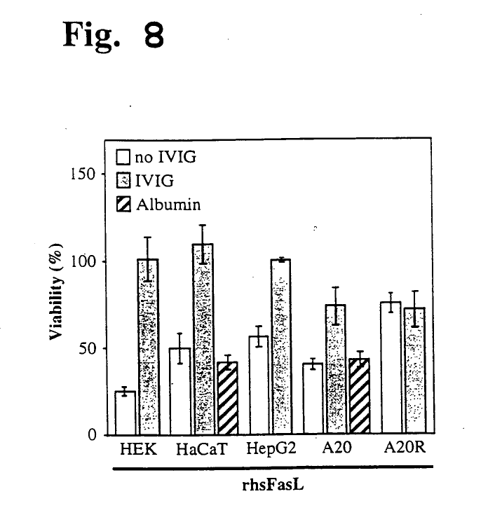

FIG. 8 is a bar graph that reflects the protective function of intravenous immunoglobulin (IVIG) mixtures on different cell lines (HEK=primary human keratinocytes; HaCaT=human keratinocytes; HepG2=hepacarcinoma cells; and A20=Fas sensitive lymphoblastoid cell lines) that occurs after an addition of apoptosis-causing rhsFasL. The percentage of the cells preserved against apoptosis (viability) has been measured with respect to the viability of the cells in absence of rhsFasL. The results of comparative tests without IVIG (white bars) and with albumin (hatched bars) (for the assessment of unspecific reactions) are likewise given.

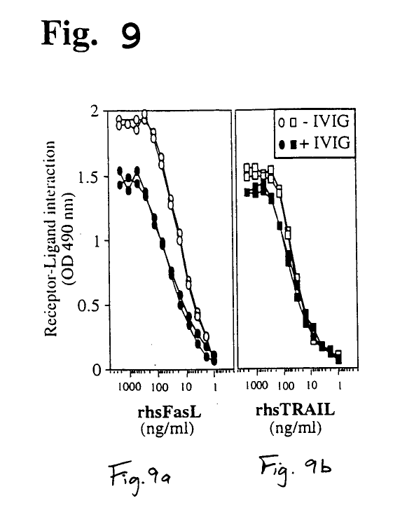

FIGS. 9a and 9b are a pair of graphs that describe the effect of IVIG mixtures on the binding of FasL to Fas (9a) and of TRAIL (another ligand causing apoptosis) to the receptor TRAIL R2 (9b). FIG. 9a shows the inhibiting effect of IVIG mixtures on the binding of FasL to Fas.

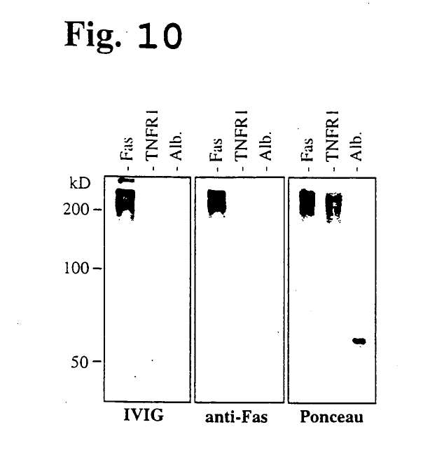

FIG. 10 is a representation of an immunoblot which shows that IVIG mixtures react positively in relation to Fas, but show only a weak reaction in relation to TNFR1 (left-hand image). Anti-Fas and Ponceau images are control experiments. The tested IVIG mixtures exhibit Fas antibodies.

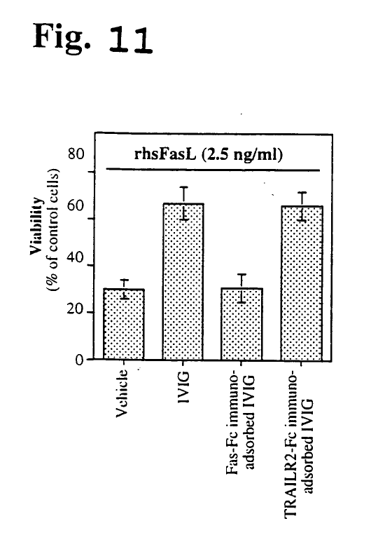

FIG. 11 is a bar graph showing the viability (as a percentage of the control cells) against rhsFasL after pre-incubation with carriers, IVIG, Fas Fc fusion protein, or TAIL2 Fc immuno-absorbed IVIG mixture (as a control). Again, the IVIG mixtures extensively preserve the A20 cells against apoptosis. Possible anti-TRAIL 2 antibodies have no importance for the protective function of the IVIG mixture, as is shown in the control experiment.

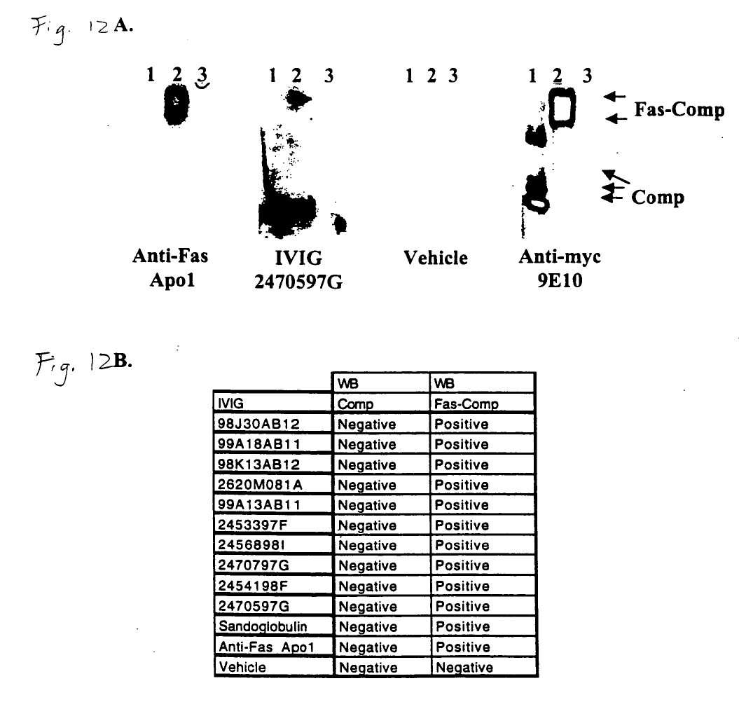

FIGS. 12a and 12b are a series of immunoblots and a table showing positive and negative results with a variety of IVIG samples tested with a Fas-Comp (cartilage oligomeric matrix protein) fusion protein. The results show that the IVIG samples contained specific anti-FasL antibodies that did not bind to Comp alone.

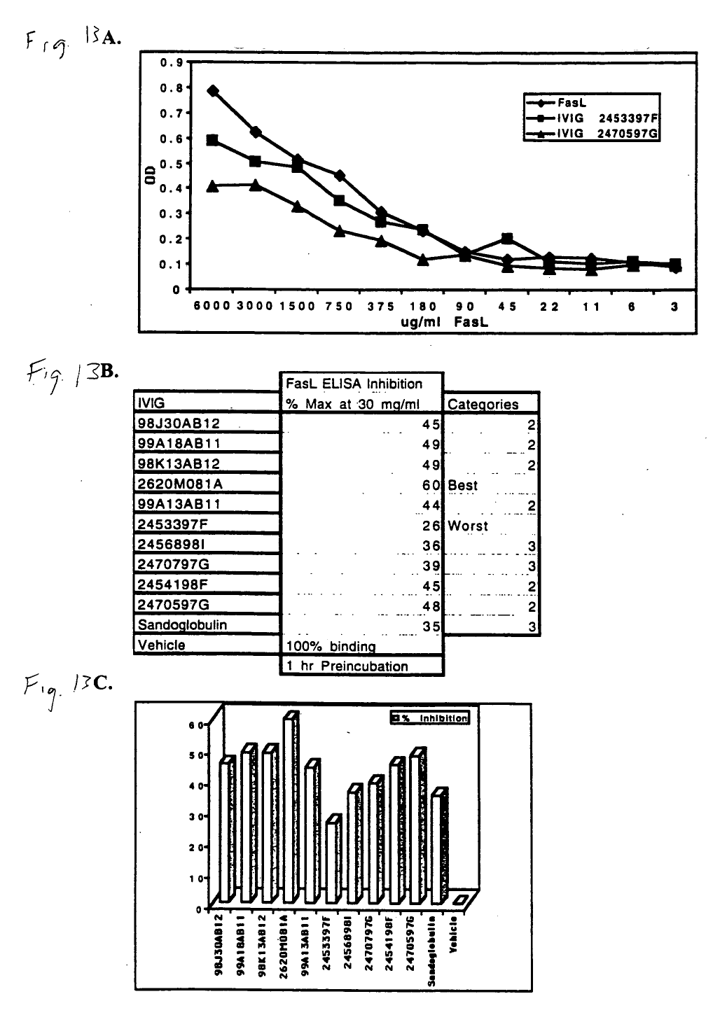

FIGS. 13a, 13b, and 13c are a graph, table, and bar graph, respectively, that show IVIG inhibition of FasL binding to Fas, and variations in the percentage of binding inhibition of various IVIG preparations.

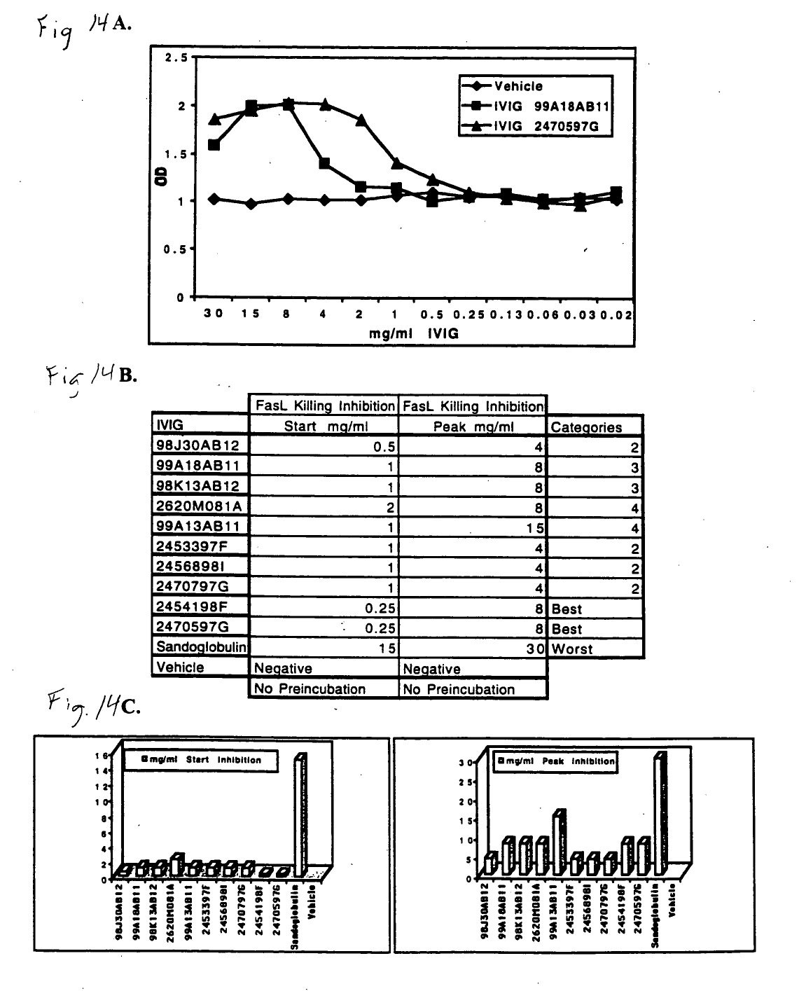

FIGS. 14a, 14b, and 14c are a graph, table, and bar graph, respectively, that show IVIG inhibition of FasL killing of A20 target cells, and variations in the percentage of killing inhibition of various IVIG preparations.

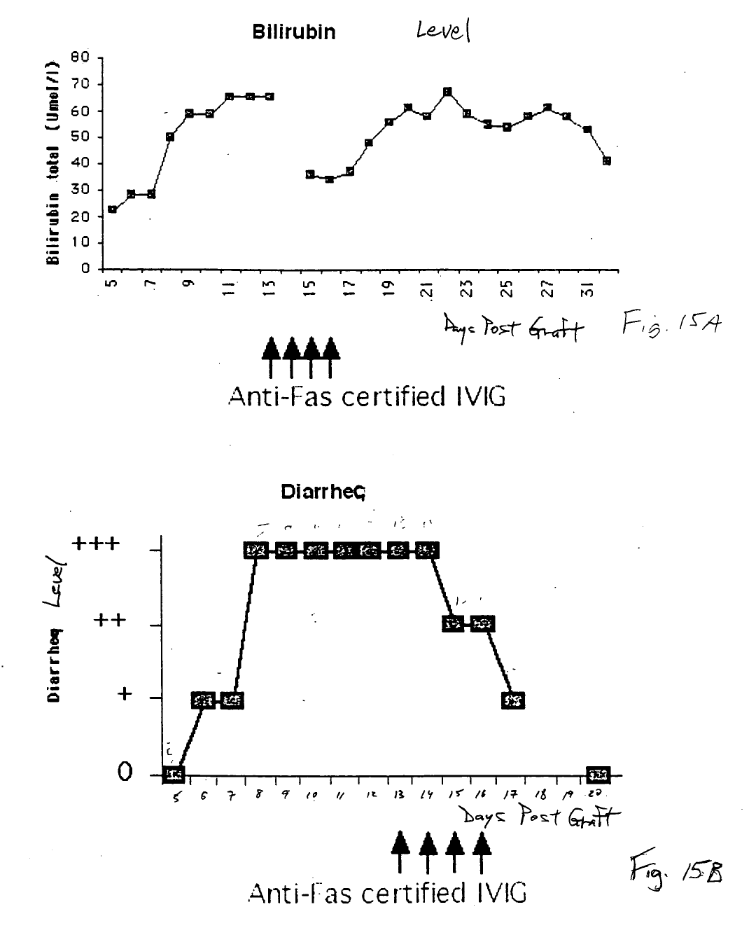

FIG. 15a is a graph showing bilirubin levels in a leukemia patient after an allogenic bone marrow transplant on Day 0. Bilirubin levels increased significantly from Day 5 to Day 13, when a FasL antibody containing IVIG composition was administered. By Day 15, the bilirubin levels dropped significantly. Thereafter, the level fluctuated and returned to normal levels.

FIG. 15b is a graph showing diarrhea levels in a leukemia patient after an allogenic bone marrow transplant on Day 0. Diarrhea reached severe levels at Day 8 and remained severe until Day 13, when a FasL antibody containing IVIG composition was administered. By Day 15, the diarrhea was less severe, and returned to normal by about Day 20.

### DETAILED DESCRIPTION

The inventors have shown that the use of compositions that contain certain antibodies oriented against Fas (anti-Fas antibodies) are suited to the treatment of human or animal conditions with an excessive apoptotic reaction, when this increased apoptotic reaction is based on increased extracellular FasL-titers (soluble and/or membrane-bound FasL). Such compositions can then be used for the manufacture of a drug for the treatment of syndromes with the above-named etiology. Compositions that contain the anti-Fas antibodies inhibiting the FasL/Fas receptor interaction prove to be particularly suitable when they are used in the treatment of toxic epidermal necrolysis (Lyell's Syndrome), graft-versus-host disease (GVHD), hepatitis, fulminant hepatitis, autoimmune thyroiditis (Hashimoto's thyroiditis), malignant tumor illnesses (e.g., melanoma), or HIV.

It was established according to the invention that, for the above-named illnesses, substantially increased extracellular FasL titers (soluble and/or membrane-bound FasL) are at least responsible; these titers destroy sensitive human cells through apoptosis. The compositions contain antibodies inhibiting the FasL/Fas receptor interaction, in particular anti-Fas antibodies. As regards these compositions, it is a matter of natural compositions, in particular blood products.

In the case of toxic epidermal necrolysis (TEN), a connection has been established for the first time in accordance with the invention to an etiology based on failed apoptotic regulation. As concerns the invention, the aforementioned illness is based on an apoptotic destruction in huge numbers of epidermal cells. This leads in the end to a separation of dermis and epidermis with fatal results in approximately 30% of the involved cases (Roujeau, Stern, New England Journal of Medicine, 331, 1272-1285, 1994). While normally the apoptosis of keratinocytes seldom occurs in the epidermis, the apoptosis of these cells is heavily increased in patients with toxic epidermal necrolysis. It has been established in the invention that uncommonly high titers of soluble FasL exist in the corresponding serums in patients with TEN. It is further known from different publications (Gutierrez-Steil at al., Journal Clinical Investigations, 101, 33-39, 1998, Berthou et al., Journal Immunology, 159, 5293-5300, 1997), that keratinocytes usually evince only small amounts of FasL. In the case of adequate stimulation, however, FasL production can be induced in keratinocytes (Guiterrez-Steil et al., Journal Clinical Investigations, 101, 33-39, 1998). In the course of the invention it has also been shown that keratinocytes from TEN patients exhibit a high FasL quantity, while healthy control persons were inconspicuous in this respect.

It has also been discovered that intravenous immunoglobulin (IVIG), produced as a blood product from “pooled” plasma from healthy donors, which until now has been used successfully only in individual cases to treat inflammatory or autoimmune illnesses, also can be used to treat human or animal pathophysiological conditions associated with increased extracellular FasL concentration (soluble and/or membrane-bound FasL), and in particular the disorder TEN. IVIG is available commercially, e.g., Sandoglobulin® from Sandoz Pharmaceuticals and various IVIG mixtures from Baxter.

Further examinations in relation to the invention have shown that a reaction of IVIG-mixtures in the aforementioned illnesses that are based on increased FasL titers can be observed when the IVIG mixtures contain antibodies that block the interaction of Fas with the extracellular ligands of FasL (soluble and/or membrane-bound).

However, only certain IVIG mixtures, as a natural blood product, have the quality which allows them to be used pharmaceutically in an effective manner for the treatment of those illnesses or health disturbances that can be traced back to increased extracellular FasL titers (soluble and/or membrane-bound). As demonstrated by the invention, sufficient anti-Fas antibody titers inhibiting the FasL/Fas interaction must be present in the IVIG mixture. To be able to conduct early suitability and quality control of natural blood plasma products, immunological processes have been established according to the invention that allow a determination of the anti-Fas antibody titers in any pharmaceutical composition, but in particular in IVIG mixtures. Only after such a suitability and quality control process can such compositions be used for the production of a drug to treat illnesses that are based on increased extracellular FasL concentrations (soluble and/or membrane-bound). Significantly, only batches or samples that have tested immunologically positive are used for treatment, while, after the in vitro test with the new procedures, batches without anti-Fas antibody activity are rejected, and not used for treatment or for the production of a drug for treatment.

In addition to specific IVIG mixtures having only slight specificity for use as a drug for the treatment of illnesses with high extracellular FasL titers (soluble and/or membrane-bound), the invention also provides a specific composition, in particular a suitable blood product, that is tested with regard to its suitability and quality and produced through procedures in accordance with the present invention.

The anti-Fas antibody titer in the composition can be quantified to guarantee a specific and sufficient dose of the composition that contains anti-Fas antibodies; the dose is dependent on the respective extracellular FasL titer in the patient. Such quantitative determinations can be undertaken with the help of a dosage graph; doses of the composition can be determined, for example, in which 50%, 75%, 85%, 90% or more, of the cells can be preserved against the apoptosis caused by FasL.

As far as the invention is concerned, for the purpose of prophylactic suitability and quality control of natural compositions with a quantified and functionally confirmed potential for inhibiting the excessive apoptosis reaction, the appropriate procedures are those in particular that allow the measurement of receptor/ligand reactions. First, in a procedural step (a), Fas, in particular its extracellular domain, is used as a ligand in the test system; a Fas-Fc fusion protein is preferred, more specifically, that will be incubated with compositions, IVIG mixtures for example, suited potentially for the treatment of the above-named illnesses. In addition, the labelled FasL will be applied to the solution previously incubated according to procedural step (a), and the portion of FasL bound to Fas or for example to Fas-Fc fusion protein will finally be determined using physical or chemical methods. Particularly appropriate are spectroscopic methods, or more specifically spectroscopic procedures that are based on the absorption of fluorescence in the visible or short UV range.

All methods for proceeding that are familiar are available here to those skilled in this field, e.g., a direct marking of the FasL with chromophores or antibodies directed against FasL that then can be marked or labelled in turn, for example, with chromophores. The antibodies marked and directed against FasL can therefore detect natural epitopes of FasL or can be directed against epitopes or markers on the FasL protein, namely introduced recombinant portions (e.g., against the so-called flag sequence).

As a further adequate immunological process, for the suitability and quality control of compositions, in particular of IVIG mixtures, of the above-mentioned kind of test system, a test system will be made available that is based on cell-destruction caused by the FasL/Fas. Cells from Fas sensitive cell lines (e.g., Jurkat cells or the lymphoblastoid cell line A20) are pre-incubated with the composition that is to be examined and tested for suitability; in addition, these cells are applied to this pre-incubated preparation of FasL (for example, recombinant human soluble FasL). The number of Fas sensitive cells that are destroyed through the FasL/Fas interaction in the course of apoptosis are determined. If the examined composition contains anti-Fas antibodies that block the FasL/Fas transduction mechanism, then Fas sensitive cells will be preserved against apoptosis by the composition. In this way, a composition is shown to be suitable for use in the production of a drug that will be used to treat the above-named health disturbances. Control experiments with cells resistant to apoptosis caused by Fas/FasL avoid a misinterpretation of the positive results.

To determine the extent of the apoptosis caused by FasL activity among the Fas sensitive cells, an Annexin-FITC cell destruction test can be used, for example, such as one sold by the Alexis Corporation, San Diego, USA, or an assay for the determination of cell viability, e.g., as sold by Boehringer, Mannheim, Germany, with the ID number WST-1.

In addition, immunoblotting methods are effective as a process for the prophylactic suitability and quality control of potentially effective pharmaceutical compositions for the treatment of the above-named illnesses. In these methods, extracellular fragments of Fas are brought into contact with the compositions, e.g., IVIG mixtures, that contain, or may contain, anti-Fas antibodies, and then, the portions of the composition linked to Fas, e.g., the anti-Fas antibodies contained in the composition, are identified through secondary antibodies that bind specifically to these.

For identification, the secondary antibodies can be labelled using familiar techniques. It has been shown that aside from marking with chromophores, a very suitable marking can be made with enzymes that shift substrata quantifiably and measurably. Such enzymes used for marking, can include, e.g., horseradish-peroxidase. In this process, those batches of the composition (IVIG mixtures or other blood products, for example) that can be used for treatment of the above-named health disturbances can be positively determined through the markings on the secondary antibody.

The procedures described above for the prophylactic suitability and quality control of compositions, in particular natural blood products, but above all IVIG mixtures, that are effective for the treatment of health disturbances or illnesses with increased FasL titers (soluble and/or membrane-bound), can also be elements of a process that contribute to the processing or improvement of these compositions. The goal here is to make compositions available, in particular natural blood products, with increased pharmaceutical effectiveness. The compositions, for example blood products, and in particular IVIG mixtures, will first be fractionated in a processing stage (a). This fractionation stage occurs in a known manner. In a further step (b), the fractions are examined for their suitability and quality, i.e., for the existence of anti-Fas antibodies. The procedures described herein are thereby executed, e.g., a receptor/ligand binding test, blotting techniques, and/or cell destruction assays. These are used alone or in combination to determine anti-Fas antibody titers in each fraction. Consequently, those fractions that have been positively tested on anti-Fas antibodies with the help of the above mentioned immunological processes (process step (c)) are isolated.

Finally, in stage (d), those fractions are concentrated so that pharmaceutically effective anti-Fas antibodies inhibiting FasL/Fas interaction are also concentrated. The product of this process exhibits an increased anti-Fas antibody concentration, possesses an increased pharmaceutical effectiveness, and can be used as a drug for the treatment of health disturbances or illnesses with increased extracellular FasL titers (soluble and/or membrane-bound). Alternatively, this product can be used to produce a drug for the treatment of such health disturbances. The scope of application for such drugs comprises both human and veterinary medicine.

In a preferred example, step (d) of the previously described procedure for the manufacture of a drug is executed in such a way that the anti-Fas antibody is contained in a purified form from the original composition, which in particular will be an IVIG mixture. Chromatographic processes that exploit the affinity between antigens and antibodies are particularly amenable for purification. Column chromatographic processes, in which the antigen, here, the Fas protein (even more advantageous would be a Fas fusion protein) is coupled to the carrier, are quite useful. The anti-Fas antibodies, for example, those bound to the Fas fusion protein, are finally eluted from the column in a known, standard process, e.g., using a salt solution (step (e)).

Through two or more, if necessary, repetitions of such a purification step, preferably combined with other biochemical purification procedures, the anti-Fas antibodies that are suited to use in the manufacture of a drug can be further purified.

In another preferred example, the procedure for the manufacture of a drug for the treatment of human or animal illnesses with increased extracellular FasL titers (soluble FasL and/or membrane-bound FasL) is modified in such a way that a highly specific drug is made available as a result. The composition, as described above, purified beforehand in large part by the chromatographic methods, or in particular the “pooled” blood product, is further processed in such a manner that only anti-Fas antibodies will be used as a drug that binds to specific epitopes in the Fas protein. Thus, anti-Fas antibodies stimulating apoptosis, in that case, may be prevented from being contained as a product of the purification, production, or preparation process for later use as a drug. This goal can be reached if one or more affinity chromatographic step(s) is (are) used for the elution of the Fas antibodies according to step (e).

On the carrier material, selected epitopes of the Fas protein that are particularly suitable for inhibiting the FasL/Fas binding are coupled to the carrier as ligands. As a result, an epitope-specific anti-Fas antibody subfraction is isolated through steps (a) to (g). In detail, a step in the process is set up after the elution (according to step (e)), in which the eluate obtained according to step (e) is passed through one or more affinity chromatographic columns. On this column (these columns), epitopes are coupled to the carrier material that bind any possible antibodies that are directed against these. The epitopes correspond to partial sequences of the Fas protein, so that anti-Fas antibodies are isolated specifically from the eluate according to (e). A highly specific drug is therefore provided for the treatment of illnesses with increased extracellular FasL titers (soluble and/or membrane-bound), in particular TEN.

This anti-Fas antibody sub-fraction obtained through the procedural steps (a) to (g) will be best prepared galenically, in that the isolated anti-Fas antibodies will be used according to their sequencing with current procedures in gene technology in humanized form. This step is therefore necessary if the composition that is basic to the process does not represent a human blood product, but is rather of animal origin, for example. The products from the procedures described above will be used for the production of a drug for the treatment of human or animal conditions with pathophysiologically increased extracellular FasL titers (soluble and/or membrane-bound), in particular for the treatment of toxic epidermal necrolysis, graft-versus-host disease, hepatitis, fulminant hepatitis, auto-immune thyroiditis, illnesses with malignant tumors or HIV.

For the treatment of the above named illnesses and/or health disturbances, monoclonal antibodies that are directed against Fas and work to inhibit the cell apoptosis caused by FasL/FasR can be considered. Such monoclonal antibodies are preferably of human origin or are of non-human origin and then humanized though a process of gene technology. The production of these antibodies can also occur through processes that are not based on the isolation from naturally appearing compositions (as for example blood products), but rather through standard and well-known procedures, for example, through methods of combinatorial chemistry for antibody production. Anti-Fas antibodies with corresponding inhibiting potential against apoptosis are identified through isolation methods and biological testing systems described herein.

### EXAMPLES

The invention is further described in the following examples, which do not limit the scope of the invention described in the claims.

### Example 1

It has been shown according to the invention that Lyell's Syndrome (TEN) is based in its etiology on increased soluble FasL titers in the serums of affected patients. Blood serums of clinically conspicuous patients were compared with those of healthy control persons. At the core of the clinical conspicuousness of TEN patients was the observation that these patients suffer from related, mostly dark-colored erythema, and a spontaneous detachment of the epidermis (from the dermis) can then be observed, exactly as with mucous-erythema and sores. The clinically conspicuous epidermal detachment has been confirmed through histological findings in all TEN patients. In all examined TEN patients, the surface of the erythemal and detached epidermis amounted to 60% or more of the total body surface (FIG. 1).

Evidence that the serum of examined patients contained soluble FasL-fractions was provided by ELISA. Results showed a 100% correlation between high FasL titers in TEN patients and almost non-detectable titers in healthy control persons (FIG. 2). Serum aliquots were taken from patients with fully formed TEN and healthy control patients and were examined with a sFasL ELISA kit (Medical & Biological Laboratories Ltd, with use of anti-FasL antibodies mAb 4H9 and 4H5). The healthy control patients were younger than 40 and without evidence of skin or systemic illnesses.

FIG. 2 illustrates the characteristically increased serum titers of soluble FasL (sFasL) in TEN patients. The sFasL concentrations are above 0.5 ng/ml in their serums. On the other hand, the serum concentrations of healthy control persons or patients with another skin condition (MPR: macular-popular skin rash), who have been used likewise as negative controls, have insignificant sFasL titers.

It has been further shown in accordance with the invention that with TEN patients, an apoptosis of keratinocytes precedes the clinically conspicuous detachment of the epidermis (FIG. 3). Here, it is a question of an early occurrence of this system manifesting itself morphologically.

FIG. 4 shows an immunoblot of monoclonal antibodies directed against FasL. Lane 1 contains a 293 pseudo-transferred cell lysate (no binding), while lane 2 contains a 293 transferred cell lysate (binding) . The antibody reaction is specifically directed against the correspondingly transferred cells.

To determine whether, and, if so, in what manner, there is a connection between keratinocyte apoptosis and conspicuous FasL serum titers, the Fas and FasL expressions were examined in skin tests of TEN patients (N=7) and compared with those of healthy control persons (N=5) (FIGS. 5a-f) . Skin biopsies were taken from TEN patients from the bordering layer between detached and not-detached skin. The biopsies were either frozen in liquid nitrogen or fixed in para-formaldehyde (4%). The immunohistochemistry was executed on the cryogenic segments, as described in French et al., J. Cell Biol., 133, 335-343, 1996.

Monoclonal anti-Fas antibodies (All, Alexis Corp., described in Hahne et al., Science, 274, 1363-1366, 1996) and monoclonal anti-Fas antibodies (UB2, immunotech) and isotope control antibodies were used. The histological skin samples from TEN patients showed a strong keratinocyte apoptosis expression, an expression after immunohistochemical proof with anti-Fas antibodies (mAb A11), a FasL expression strongly increased in comparison to healthy control persons and in comparison with healthy control persons a nearly unchanged Fas expression in the cell surface.

Specifically, FIGS. 5a-f show samples of skin tissues of healthy persons (FIGS. 5a and d) and from TEN patients (5b, c, e, and f) that have been treated with anti-Fas antibodies. As a control, control antibodies have been used that then exhibit the unspecific antibody reaction (5c and f). In FIG. 5b, the strong antibody reaction against FasL in the epidermis of TEN patients can be clearly seen.

Finally, it has been shown for TEN in accordance with the invention that the apoptosis of keratinocytes characteristic for TEN is caused in fact by the FasL/Fas signal transduction path. The lytic activity of cutaneous FasL has been examined in vitro. Frozen samples of the skin from healthy control persons (N=5) and TEN patients (N=3) were brought into contact with Fas sensitive Jurkat cells for a period of six hours and then the apoptosis of Jurkat cells was determined with the help of an Annexin FITC test or a Cytochrome c-test. It was ascertained that the TEN skin samples destroy in a reproducible manner (both with the Annexin FITC and with Cytochrome c-assay) three to four times as many Jurkat cells as corresponding skin samples of healthy control persons. FIG. 6 shows the results (with statistical error bars) of experiments with Fas sensitive Jurkat cells that have been stratified on cryogenic skin segments from healthy, TEN, and MPR patients. The bars in the graph show the percentage of apoptotic Jurkat cells in each case after a 6 hour incubation, as determined by flow cytometry. The tissues of TEN patients incite a significantly increased apoptotic reaction.

It was further shown in the experiment that in the presence of FasL blocking antibodies (NOK 1) the cytotoxicity induced by means of the TEN skin tissue samples was completely eliminated in the Jurkat cells (FIG. 6, 4th bar in graph). The tissue samples were pre-incubated for 30 minutes with anti-FasL antibodies (NOK 1, 2.5 μg/ml, Pharmingen), then placed in the Fas sensitive Jurkat cells (human leukemia cells), as already described in Strand et al., Nature Medicine, 2, 1361-1366, 1996 or in Buchner et al., Journal Clinical Investigation, 100, 2691-2699, 1997. The portion of Jurkat cells lysed through apoptosis was measured with the help of flow cytometry with the use of Annexin FITC (Pharmingen, as described in Vermes, Haanen, Steffens-Nakken, Reutelingsperger, Journal Immunology Methods, 184, 39-51, 1995). The addition of soluble recombinant human FasL (rhs FasL) showed that human keratinocytes are apoptosis-sensitive in the presence of FasL (FIG. 7). Soluble recombinant FasL proteins labelled with FLAG (Alexis Corporation) were incubated with Jurkat cells for a period of 6 and 10 hours and then the viability was determined with the help of a proliferation test (WST-1, Boehringer, Mannheim, Germany).

### Example 2

This example demonstrates that IVIG (intravenous immunoglobulin) as a blood product that is produced from pooled plasma of healthy donors can function as an inhibitor of apoptosis caused by FasL/Fas.

Keratinocytes were incubated together with IVIG before their exposure to rhsFasL. It was noticed that in rhs-FasL concentrations that are able to induce a 75% keratinocyte apoptosis, the addition of IVIG, namely 30 mg/ml (calculated according to the daily dose for the treatment of a patient weighing 60 kg) completely inhibits the keratinocyte apoptosis related to Fas/FasL. In this experiment, keratinocytes of the cell lines HaCaT, hepatocarcinoma cells (HepG2), Fas sensitive cells A20 and Fas resistant lymphoblastoid cell lines A20R (and albumin as control) were incubated with IVIG and finally added to soluble FasL. FIG. 8 illustrates the protective function of IVIG mixtures on these different cell lines that occurs after an addition of apoptosis-inducing rhsFasL. The percentage of the cells preserved against apoptosis (viability) were measured with respect to the viability of the cells in the absence of rhsFasL. The protective function of IVIG is not limited to the previously named keratinocytes, but rather comprises all Fas sensitive cells. In the present experiment, it was proven that specifically IVIG has this effect, in that similar concentrations of albumin offer no apoptotic protection (FIG. 8).

In a further experiment to explain the working mechanism of IVIG, it was shown that its protective effect is based on an inhibition of the Fas-induced cell destruction, while the Fas receptor, but not the Fas ligand (FasL), is blocked. An IVIG mixture was pre-incubated with rhsFasL and this pre-incubated mixture was put into contact with the above-mentioned cell lines. No lytic, which is to say, apoptotic, effect could be discerned in the cells of these cell lines.

In a further experiment in the framework of this example, it was again confirmed with the help of an ELISA that IVIG mixtures inhibit the Fas/FasL interaction. Here the receptor ligand interaction was determined in relation to the presence or absence of IVIG. FIGS. 9a and 9b illustrate the effect of IVIG mixtures on the binding of FasL to the Fas receptor and the binding of TRAIL (another ligand causing apoptosis) to the receptor TRAIL R2 (FIG. 9b). The binding of the ligand to the receptor was measured in the manner described in Schneider et al., J. Exp. Med., 1197, 1-9, 1998 and Schneider et al., J. Biol. Chem., 272, 18827-18833, 1997. FIG. 9a shows the inhibiting effect of IVIG mixtures on the binding of FasL to Fas. Fi. 9b shows that the binding of soluble TRAIL to its receptor TRAIL R2 (here used as TRAIL R2 Fc fusion protein) was not blocked by the IVIG mixture.

In the case of TRAIL, it is a question of the ligand causing apoptosis; the protective function of IVIG could have been based on the binding inhibition of this ligand. This comparative experiment again shows that the FasL/Fas interaction is specifically blocked by an IVIG mixture.

### Example 3

This example provides evidence for the molecular basis for the inhibiting effect of IVIG. To show that this effect is based on the anti-Fas antibodies naturally present in IVIG compositions, an examination was undertaken to see if a portion of the IVIG mixture binds to human Fas, and then, whether an IVIG fraction that is negative with respect to anti-Fas antibodies also fails to inhibit Fas-induced apoptosis.

Immunodetection was undertaken on a gel. The IVIG mixture was tested against albumin (as a control) or against purified recombinant protein structures that exist in the extracellular domain of the human Fas (so-called Fas-comp) or the extracellular domain of the tumor necrosis factor receptor (TNFR1-comp), in each case fused with a 55 amino acid long protein linker, cartilage oligomeric matrix protein (“Comp”), which is described in Terskikh et al., Proceedings of the National Academy Science, USA, 94, 1663-1668, 1997. These experiments show that IVIG mixtures provide clearly positive signals in relation to Fas-comp, but not in relation to albumin, and only weak signals in relation to TNFR1-Comp (FIG. 10, left-hand set of lanes). The middle and the right-hand sets of lanes are control experiments. These results demonstrate that the tested IVIG mixtures contain Fas antibodies.

This suggests that at least a small anti-TNFR1-activity could be existent in IVIG mixtures. A removal of the anti-Fas antibodies from the IVIG mixture through multiple purification via Fas-Fc fusion protein affinity chromatography columns shows that through the loss of anti-Fas antibodies, both the binding ability of IVIG to Fas-comp and the blockade of the Fas-induced cell-destruction is eliminated.

FIG. 11 shows the viability (as a percentage of the control cells) against rhsFasL after pre-incubation with carriers (vehicle), IVIG, Fas Fc fusion protein (Fas-Fc immunoabsorbed IVIG), or TRAIL2-Fc immunoabsorbed IVIG mixture (as a control). Again, the IVIG mixtures extensively preserve the A20 cells against apoptosis. However, possible anti-TRAIL 2 antibodies have no importance for the protective function of the IVIG mixture, as is shown in the control experiment.

### Example 4

In an open, uncontrolled pilot study, 10 different patients with TEN were treated in three different hospitals (Geneva, Lausanne, and Bern) with IVIG; the doses for the patients used amounted to from 0.2 to 0.75 g/kg/d for a period of four successive days. In all 10 patients, the progression of TEN was interrupted quickly (within 24 to 48 hours) after the IVIG mixture was infused. Side-effects did not occur in significant amounts; on the contrary, a fast skin healing was observed (see Table 1). Therefore, the therapeutic effect of IVIG in TEN patients was shown for the first time in a clinical study according to the invention.

TABLE 1

| | | | Dose of | Time | Time to |
|---|---|---|---|---|---|
| | Erythema | | IVIG | from | response/ |
| Patient/ | (%)/ | (g/kg/d)/ | onset to | skin | |
| Age | Detachment | | duration | treatment | healing |
| (YR) Sex | (%) * | Casual Drug | (d) | (d) + | (d) § |
| 1. ME/23/M | 50/50 | Ibuprofen | 0.75/4 | 5 | 2/7 |
| 2. BG/22/F | 50/30 | Carbamazepin | 0.75/4 | 4 | 1/7 |
| 3. ER/57/F | 40/20 | Ciprofloxacin | 0.375/4 | 3 | 2/5 |
| 4. MP/11/M | 70/20 | Paracetamol | 0.75/4 | 4 | 1/6 |
| 5. BK/26/M | 20/60 | Ceftriaxon | 0.75/4 | 2 | 1/10 |
| 6. IF/88/F | 50/10 | Allopurinol | 0.2/4 | 4 | 2/5 |
| 7. FD/13/M | 60/40 | Cefuroxim | 0.45/4 | 2 | 2/9 |
| 8. HW/65/M | 60/30 | Doxycycline | 0.75.4 | 4 | 2/12 |
| 9. CM/28/F | 80/5 | Undetermin. | 0.75/4 | 4 | 1/4 |
| 10. PE/61M | 40/20 | Phenytoin | 0.75/4 | 4 | 1/4 |

Table 1 summarizes the clinical data of the 10 TEN patients who were treated with IVIG mixtures. In detail, Table 1 includes: (a) age/sex of the patients, (b) the body surface affected with erythema and epidermal detachment, (c) the medical drug trigger for TEN, (d) the administered dose of the IVIG mixture in g/kg/d and the length in days of the treatment with IVIG, (e) the time frame in days from the beginning of the clinical symptoms to the beginning of treatment, and (f) the time frame until the clinical reaction to the treatment and skin healing.

### Example 5

IVIG is described herein to contain anti-Fas Ab. To screen commercial IVIG preparations (Baxter), a series of Western blots were done. We have loaded on SDS-PAGE gels a recombinant protein composed of the ectodomain of the Fas receptor fused to a support made with the Cartilage Oligomeric Matrix Protein (Comp). As a control, the Comp protein was used alone. After transfer to nitrocellulose, the IVIG samples were probed for recognition of the Fas-Comp protein. As shown in FIG. 12a, IVIG 2470597G specifically bound to Fas-Comp. The specificity was given by the fact that the Comp protein alone was not recognized. As positive controls, we used the anti-Fas Ab which bind to Fas-Comp and the 9E10 Ab which binds to a myc-tag peptide attached to both Comp proteins. A Western blot with vehicle alone was negative. All commercial preparations recognized Fas-Comp, and these data are summarized in FIG. 12b.

Once it was determined that the IVIG preparations contain anti-Fas Ab, they were tested for biological function using ELISA QC. Fas was coated onto plastic and, as shown in FIG. 13a, the pre-incubation with IVIG 2470597G or IVIG 2453397F at 30 mg/ml inhibited FasL binding. There were variations among the different preparations. The percentage of maximal inhibition varied between 60 and 26%, as shown in FIGS. 13b and 13c. This may signify that the preparations differed in their potential for blocking Fas-FasL interactions. The effect seen is likely due to a difference of anti-Fas Ab titer.

The second test used to evaluate IVIG was another QC test set up for FasL. It is a well established, reliable test where FasL-mediated killing of A20 target cells was measured. To run this test, we first determined what concentration of FasL would kill 50% of A20 target cells. We then titered the different IVIG preparations with that fixed concentration of FasL. As shown in FIG. 14a, the IVIG inhibited FasL-mediated killing. The inhibition was dose dependent and at high doses of IVIG, killing was completely inhibited. As for the ELISA test, there were some differences among the IVIG batches as summarized in FIGS. 14b and 14c. Some Baxter batches were superior to other commercial preparations tested.

We have found differences in the concentration of IVIG needed to start inhibiting the FasL-mediated killing as well as the concentration needed to completely block it. This is another indication that IVIG preparations have different potential for blocking FasL-Fas interaction and consequent FasL-mediated killing.

All commercial IVIG preparations tested contain anti-Fas Ab. The results of the biological tests show that all tested IVIG batches were able to inhibit Fas-FasL interaction to some degree. However, there are differences between batches. These results reflect the internal status of the anti-Fas Ab level of each individual IVIG preparation. We have not found distinct groups of IVIG in terms of beneficial vs. no effects. Therefore, we have grouped the IVIG products in four categories (see FIG. 14b). Category 1 is the best, and Categories 2 through 4 are sequentially less effective. The IVIG mixtures are categorized according to the concentration required to start inhibition of FasL-mediated killing and the peak concentration for maximal inhibition. We have also tested a another commercial preparation (Sandoz) and found it less active the Baxter preparations (no Category).

Other batches of commercial IVIG mixtures can be tested as described herein and categorized prior to use in a patient. For optimal effectiveness, with lowest dosages, IVIG mixtures in Category 1 should be used for therapy.

The isolation, purification and characterization of the immunoglobulin fraction that reacts with Fas and is most likely responsible for the beneficial effect of IVIG on patients will enable the production of anti-Fas Ig factions which can be further tested with the protocols described herein used to evaluate the IVIG. The production of an active anti-Fas fraction would allow physicians to inject into patients much lower amounts of Ig with much higher efficacy. On the other hand, the IVIG preparations depleted of anti-Fas Ab could be used for other applications where these Abs are not necessary to induce a beneficial effect in patients.

Methods

As shown in FIG. 12a, 100 ng Comp-myc (lane 1), FasComp-myc (lane 2), and the MW markers (lane 3) were loaded on as SDS-PAGe gel and proteins resolved. After transfer onto nitrocellulose, the blots were incubated with 200 μg/ml IVIG, with vehicle alone, with the anti-Fas Apol Ab or with the anti-myc 9E10 Ab at 1 μg/ml for 1 hour. A secondary HRP-conjugated Ab was then added for 1 hour. Proteins were visualized using a chemiluminescence detection system. FIG. 12b is a table summarizing the Western Blot results obtained with the different IVIG preparations. All contained some amount of anti-Fas antibodies.

As shown in FIGS. 13a to 13c, IVIG interferes with the Fas-FasL interaction. 40 ng Fas were coated per well of a 96 well plate. After blocking, IVIG was added for 1 hour at 30 mg/ml. Without washing the plates, FasL, which is Flag-tagged, was added at the indicated concentrations. After 1 hour incubation, plates were extensively washed and incubated with an anti-Flag Ab for 1 hour followed by a secondary HRP-conjugated Ab. FasL binding was detected using an OPD substrate. Plates were then read at 490 nm in an ELISA reader. FIG. 13a shows the resulting interference with the Fas-FasL interaction. FIGS. 13b and 13c summarize the ELISA results obtained with the different IVIG preparations, and show the variability of IVIG preparations form 26 to 60% binding inhibition.

FIGS. 14a to 14c show IVIG inhibition of FasL-mediated killing of A20 target cells. 50,000 A20 target cells were seeded per well of a 96 well plate. IVIG was added to reach the indicated final concentration and pre-incubated for 30 minutes at room temperature. Then 50 ng/ml FasL was added. Cells were incubated for at least 10 hours at 37° C. Viability was then assessed using the PMS/MTS method. Plates were read in an ELISA reader at 490 nm. FIG. 14a shows the results. FIGS. 14ba and 14c summarize the A20 test results obtained with the different IVIG preparations, and shown the variability of the IVIG preparations from 8 to 15 peak mg/ml killing inhibition.

### Example 6

Cytolytic T-lymphocytes (CTLs) from mice deficient in both functional Fas ligand and perforin show low residual cytolytic activity (due in part to TNF), but virtually completely lose their capacity to induce lethal acute graft-versus-host disease. Using these mice as donors, we have shown that acute lethal GVHD, induced by the transfer of splenocytes from C57BL/6 mice into sensitive BALB/c recipients, is dependent on both perforin and FasL-mediated lytic pathways. When spleen cells from mutant mice lacking both effector molecules were transferred to sublethally irradiated allogeneic recipients, mice survived. Delayed mortality was observed in animals grafted with cells deficient in only one lytic mediator (perforin or FasL).

It has been shown that although recipients of allogeneic FasL-defective donor T-cells undergo sever GVHD-associated cachexia, they only develop minimal histopathological signs of hepatic and cutaneous GVHD. On the other hand, recipients of perforin-deficient allogeneic donor T-cells developed signs of acute GVHD, but the time of onset was significantly delayed. These findings demonstrate that Fas-mediated anti-recipient cytotoxicity is implicated in the development of hepatic and cutaneous GVHD, but is not involved in the genesis of GVHD-associated cachexia.

Other groups have analyzed the expression of FasL on lymphocytes during GVHD. FasL expression on host-derived intestinal intraepithelial lymphocytes (IELs), which are known to increase in number during acute GVHD in mice, was studied. It was shown that αβ or τδ IELs, which express little or no FasL mRNA before the occurrence of GVHD, express high levels during GVHD. In addition, the latter IELs showed cytotoxicity against Fas-transfected target cells and intestinal epithelial cells, which was partially blocked by addition of Fas Fc. These results suggest therefore that Fas-mediated cytotoxicity by host-derived IELs may be partly responsible for the enteropathy that occurs during acute GVHD. Both the expression of Fas/FasL and lymphocyte subsets in patients who underwent HLA-matched/related allogeneic bone marrow transplantation (n=16) and normal donors (n=10) were studied. It was shown that there is an increase in the percentage of CD8+ cells that express Fas and FasL in patients with acute GVHD after bone marrow transplantation, providing support in humans for the previous observations made in mice, showing the donor lymphocyte FasL is directly involved in the effector phase of acute GVHD.

We recently analyzed the expression of Fas and FasL in cutaneous lesion of acute GVHD in 15 patients. Our immunohistochemical analysis revealed strong FasL expression in the dermal lymphoid infiltrate that comes into contact with the epidermis.

Taken together, recent animal and human studies of GVHD show that lymphocyte FasL, which is induced during acute GVHD, mediates the tissue damage observed clinically (skin, gut and liver), and contributes to the mortality of GVHD.

Given the above experimental evidence suggesting the Fas-mediated cell death is implicated in the pathogenesis of acute GVHD, and knowing that Fas-mediated cell death can be inhibited by anti-Fas antibodies contained in IVIG, we started a pilot clinical study to investigate whether the administration of IVIG mixtures pre-tested for anti-Fas activity are able to modify the evolution of acute GVHD. Currently, treatments of acute GVHD are mainly based on the administration of immunosuppressive drugs, which can be detrimental. The use of a treatment potentially able to protect targets form unwanted immunological attack could be of considerable clinical benefit.

Results

The first patient in the clinical study achieved a highly significant benefit from the IVIG therapy according to the present invention.

The first patient was a 40 year old woman with acute leukemia (LMA M5) in complete remission after chemotherapy. She received an allogeneic bone marrow transplant (HLA compatible, T-depleted) on day 0, and despite GVHD prevention with methotrexate and cyclosporin, she developed acute Grade III GVHD by day 11. The acute GVHD consisted of a skin rash covering 70% of the body surface, perturbed liver functions (increased bilirubin levels at 54 μmol/l), and severe gut involvement (1500 ml of diarrhea per day). See FIGS. 15a and 15b.

On day 13 she was given 0.75 g/Kg/day of Fas-certified IVIG (tested and certified according to the present invention) by perfusion for four consecutive days. The IVIG used in this case was tested for anti-Fas activity (certified) prior to treatment and was shown to provide maximal inhibition of Fas-mediated cell death (of A20 cells), at a concentration of 8 mg/ml. This is important, considering that several IVIG batches tested so far were not as active in the same functional assay.

As shown in FIGS. 15a and 15b, by day 15, i.e., at the end of the second day of IVIG treatment, an important and significant decrease in the objective measures of liver and gut dysfunction occurred. The bilirubin levels had returned virtually to normal values (FIG. 15a), and the volume of diarrhea decreased from 1500 ml/day to less that 1000 ml/day. Within a week of treatment initiation there was no more diarrhea, and digestive function had virtually normalized (FIG. 15b). Following IVIG treatment, the skin rash also progressively decreased, to totally disappear by 10 days after the initiation of treatment. The patient left the hospital 3 weeks after the initiation of IVIG treatment without any signs of GVHD.

In conclusion, the effect of the treatment of this patient with Fas-certified IVIG on objective markers of acute GVHD activity was spectacular, being both rapid and clearly effective on reversing disease in the 3 main target organs (skin, liver, and gut).

Conclusion

Based on the pilot experimental data provided herein, it appears that IVIG preparations that contain significant levels of anti-Fas antibodies (for example, in this particular case, the IVIG had a maximal inhibitor activity at a concentration of 8 mg/ml by functional testing as described herein) are able to rapidly block the cutaneous, hepatic, and digestive involvement in acute GVHD.

Taken together with the recently published work on pathogenesis of acute GVHD, our novel data showing that lymphocytes in the skin of patients with acute cutaneous GVHD strongly express FasL, and the above therapeutic effect of anti-Fas certified IVIG in GVHD, it appears that selective inhibition of human Fas (by anti-Fas certified IVIG, Fas-comp, or humanized or human monoclonal antagonistic anti-Fas antibodies) is a novel therapeutic strategy for the treatment of GVHD.

Formulations

The IVIG mixtures and compositions can be formulated as a solution or suspension, as well as a tablet, solid drug composition, or an ointment or cream that can penetrate the skin. In the preparation of these compositions, at least one pharmaceutical carrier can be included. Examples of pharmaceutical carriers may include one or more solvents (e.g., water or physiological saline), solubilizing agents (e.g., ethanol or polysorbates), agents for achieving isotonicity, preservatives, antioxidizing agents, excipients (e.g., lactose, starch, crystalline cellulose, mannitol, maltose, calcium hydrogen phosphate, light silicic acid anhydride, or calcium carbonate), binders (e.g., starch, polyvinylpyrrolidone, hydroxypropyl cellulose, ethyl cellulose, carboxy methyl cellulose, or gum arabic), lubricants (e.g., magnesium stearate, talc, or hardened oils), or stabilizers (e.g., lactose, mannitol, maltose, polysorbates, macrogols, or polyoxyethylene hardened castor oils). If necessary, glycerin, dimethylacetamide, 70% sodium lactate, a surfactant, or a basic substance such as sodium hydroxide, ethylenediamine, ethanolamine, sodium bicarbonate, arginine, meglumine, or trisaminomethane is added. Standard pharmaceutical preparations such as solutions or dispersions can be formed with these components. If the composition is administered orally, flavorings and colors can be added.

The concentration of the IVIG mixture or Fas antibodies in the compositions of the invention will vary depending upon a number of factors, including the dosage to be administered, and the route of administration.

Administration

The compositions of the invention can be administered by parenteral administration, for example, intravenous, subcutaneous, intramuscular, intraorbital, ophthalmic, intraventricular, intracranial, intracapsular, intraspinal, intracisternal, intraperitoneal, topical, intranasal, aerosol, scarification, and possibly also oral, buccal, rectal, vaginal, or topical administration. The compositions of the invention may also be administered by the use of surgical implants which release the compounds of the invention. Bolus injections or infusions are suitable.

In general terms, the compounds of the invention can be provided in an aqueous physiological buffer solution containing about 0.1 to 10% w/v compound for parenteral administration, typically after determining that a subject has a disorder associated with increased extracellular levels or titers of FasL as described herein. General dosage ranges to be administered will depend upon the type and extent of progression of the disorder being addressed, the overall health of the patient, and the route of administration.

Other Embodiments

It is to be understood that the foregoing detailed description is intended to illustrate and not limit the scope of the invention, which is defined by the scope of the appended claims. Other aspects, advantages, and modifications are within the scope of the following claims.

What is claimed is:

1. A method of treating a subject having a disorder associated with increased extracellular Fas ligand titers, the method comprising administering to the subject a composition comprising anti-Fas antibodies in an amount effective to inhibit binding of Fas ligands to Fas receptors in the subject.

2. The method of claim 1, wherein the disorder is toxic epidermal necrolysis (Lyell's Syndrome), graft-versus-host disease (GVHD) , hepatitis, fulminant hepatitis, autoimmune thyroiditis (Hashimoto's thyroiditis), malignant tumor illnesses (e.g., melanoma), or HIV.

3. The method of claim 1, wherein the disorder is toxic epidermal necrolysis.

4. The method of claim 1, wherein the disorder is graft-versus-host disease.

5. The method of claim 1, wherein the composition comprises an intravenous immunoglobulin (IVIG) mixture.

6. The method of claim 5, wherein the IVIG is of human origin.

7. The method of claim 5, wherein the composition contains a level of anti-Fas antibodies sufficient to inhibit at least 40 percent of FasL binding to Fas receptor.

8. The method of claim 5, wherein the composition contains a level of anti-Fas antibodies sufficient to inhibit at least 50 percent of FasL binding to Fas receptor.

9. The method of claim 5, wherein the composition is administered at a dosage of at least 0.1 g/kg/day.

10. The method of claim 5, wherein the composition is administered by infusion.

11. The method of claim 10, wherein the composition is administered at a dosage of at least 0.1 g/kg/day.

12. The method of claim 4, wherein the composition is administered by infusion at a dosage of at least 0.75 g/kg/day.

13. A method of treating a subject having graft-versus-host-disease (GVHD), the method comprising administering to the subject a composition comprising anti-Fas antibodies in an amount effective to inhibit binding of Fas ligands to Fas receptors in the subject.

14. The method of claim 13, wherein the composition comprises an intravenous immunoglobulin (IVIG) mixture.

15. The method of claim 14, wherein the IVIG is of human origin.

16. The method of claim 14, wherein the IVIG contains an anti-Fas antibody at a concentration of at least 0.1 mg/ml.

17. The method of claim 14, wherein the IVIG contains an anti-Fas antibody at a concentration of at least 8 mg/ml.

18. The method of claim 13, wherein the composition comprises an anti-Fas antibody and is administered at a dosage of at least 0.1 mg/kg/day for at least two days.

19. The method of claim 14, wherein the IVIG is administered at a dosage of least 0.1 g/kg/day for at least two days.

20. The method of claim 14, wherein the IVIG is administered by infusion at a dosage of 0.75 g/kg/day for four consecutive days.

21. A method for determining the prophylactic suitability and quality control of a composition for use in treating a disorder associated with increased extracellular Fas ligand titers, the method comprising
(a) incubating the composition with a Fas-Fc fusion protein in a solution;
(b) adding to the solution a labelled Fas ligand; and
(c) detecting the amount of Fas ligand bound to the Fas-Fc fusion protein as an indication of the presence of anti-Fas antibodies in the composition, wherein an amount of anti-Fas antibodies in the composition sufficient to inhibit binding of Fas ligand to Fas receptor indicates that the composition is suitable for use in treating a disorder associated with increased extracellular Fas ligand titers.

22. The method of claim 21, wherein the composition is an intravenous immunoglobulin (IVIG) mixture.

23. The method of claim 21, wherein the percentage of binding inhibition is at least 40 percent.

24. The method of claim 21, wherein the amount of bound Fas ligand is determined chemically or physically.

25. A method for determining the prophylactic suitability and quality control of a composition for use in treating a disorder associated with increased extracellular Fas ligand titers, the method comprising
(a) incubating Fas sensitive cells with the composition in a solution;
(b) adding soluble Fas ligand to the solution; and
(c) determining the percentage of Fas sensitive cells in which apoptosis is inhibited compared to cells not incubated with the composition, wherein a composition that inhibits apoptosis of Fas sensitive cells is suitable for use in treating a disorder associated with increased extracellular Fas ligand titers.

26. The method of claim 25, wherein the composition is an intravenous immunoglobulin (IVIG) mixture.

27. The method of claim 25, wherein the percentage of inhibition of Fas sensitive cell apoptosis is at least 40 percent.

28. A method for determining the prophylactic suitability and quality control of a composition for use in treating a disorder associated with increased extracellular Fas ligand titers, the method comprising
(a) combining Fas receptors with the composition;
(b) adding labelled secondary antibodies that bind specifically to anti-Fas antibodies; and
(c) detecting the labelled secondary antibodies as an indication of the presence of anti-Fas antibodies bound to the Fas receptors, wherein the presence of anti-Fas antibodies in the composition indicates that the composition is suitable for use in treating a disorder associated with increased extracellular Fas ligand titers.

29. The method of claim 28, wherein the Fas receptors and the composition are combined in a Western blot technique.

30. The method of claim 28, wherein the composition is an intravenous immunoglobulin (IVIG) mixture.

31. A method of preparing a drug to treat disorders associated with increased extracellular Fas ligand titers, the method comprising
(a) fractionating a composition;
(b) examining each fraction to determine the presence of anti-Fas antibodies;
(c) isolating each fraction that contains anti-Fas antibodies; and
(d) concentrating the isolated fractions for use as the drug.

32. The method of claim 31, wherein the composition is an intravenous immunoglobulin (IVIG) mixture.

33. The method of claim 32, further comprising
(e) purifying and isolating the anti-Fas antibodies in the isolated fractions by affinity chromatography.

34. The method of claim 33, wherein the affinity chromatography comprises the use of column chromatography using Fas fusion proteins bound to the column.

35. The method of claim 33, wherein the affinity chromatography comprises the use of one or more chromatographic columns, each column having linked thereto a specific amino acid sequence of the Fas fusion protein that corresponds to a specific Fas antibody epitope, wherein all Fas antibody epitopes are bound to the one or more columns and are then eluted.

36. A composition for the treatment of disorders associated with increased extracellular Fas ligand titers, the composition comprising anti-Fas antibodies that inhibit binding of Fas ligand to the Fas receptor.

37. The composition of claim 35, wherein the anti-Fas antibodies are of non-human origin and are humanized.

38. The composition of claim 35, wherein the composition comprises an intravenous immunoglobulin (IVIG) mixture from a human.
